(19) United States
(12) Patent Application Publication
(10) Pub. No.: US 2021/0047249 A1
(43) Pub. Date: Feb. 18, 2021

(54) Process for Transalkylation of Aromatic Fluids

(71) Applicant: ExxonMobil Chemcial Patents Inc., Baytown, TX (US)

(72) Inventors: Tan-Jen Chen, Seattle, WA (US); Wenyih F. Lai, Bridgewater, NJ (US); Brett T. Loveless, Houston, TX (US); Matthew S. Ide, Doylestown, PA (US); Jeevan S. Abichandani, Houston, TX (US)

(21) Appl. No.: 16/071,408

(22) PCT Filed: Feb. 10, 2017
(86) PCT No.: PCT/US2017/017300

§ 371 Date: Jul. 19, 2018

Related U.S. Application Data
(60) Provisional application No. 62/313,993, filed on Mar. 28, 2016.

(30) Foreign Application Priority Data
May 19, 2016 (EP) 16170167.5

(51) Int. Cl. C07C 6/12; B01J 29/70

(52) U.S. Cl. CPC C07C 6/126; C07C 2529/18; B01J 29/70; B01J 29/7038

(57) ABSTRACT

Systems and methods are provided for an improved transalkylation process that better tolerates the presence of C₁₀₊ aromatics and may be conducted substantially in the liquid phase. The transalkylation feedstock may comprise alkyl-substituted benzenes and naphthalene and the transalkylation effluent comprises alkyl-substituted naphthalene and benzene, toluene, and/or xylenes.

### PRIORITY

This application claims priority to and the benefit of U.S. Provisional Application No. 62/313,993, filed on Mar. 28, 2016, and EP Search Application 16170267.5, filed May 19, 2016, the disclosures of which are incorporated herein by reference in their entireties.

### TECHNICAL FIELD

Systems and methods are provided for transalkylation of alkylated mononuclear aromatic compounds in the presence of polycyclic aromatic compounds.

### BACKGROUND

Production of xylenes from mixed aromatic streams is an important process commercially. Due to various equilibria, aromatic formation processes can tend to produce relatively low amounts of xylenes relative to other single ring aromatics. One option for converting a mixed feed of aromatics to produce additional xylenes is to perform a transalkylation process. Conventional transalkylation processes are typically performed under gas phase conditions, including temperatures of at least about 380° C., and include exposing benzene and/or toluene and C₉₊ aromatics to a transalkylation catalyst. However, the presence of C₁₀₊ aromatics in the transalkylation feed can have a negative impact on the catalyst life. C₁₀₊ aromatics commonly found in mixed aromatic streams are trimethylbenzenes, indane, diethylbenzenes, methylpropylbenzenes, dimethylethylbenzenes, tetramethylbenzenes, methylindanes, and naphthalene. Additionally, the presence of ethylbenzene in aromatic streams can have a negative impact on downstream separations or production of xylenes.

U.S. Pat. No. 7,241,930 describes methods for transalkylation of aromatic fluids. Aromatic fluids are fluids containing a variety of aromatic compounds, including alkylated monocyclic aromatic compounds and polycyclic aromatic compounds. Transalkylation can be performed by exposing an aromatic fluid to an acidic zeolite catalyst, optionally in the presence of hydrogen.

It would be desirable to improve transalkylation processes involving C₁₀₊ aromatics and convert undesirable compounds into products with enhanced value and recover by-products for recycling. Further, performing transalkylation on a feed that is at least partially in the liquid phase would be desirable to minimize energy consumption.

### BRIEF SUMMARY

At least some embodiments disclosed herein are directed to a transalkylation process that uses a different catalyst than that used in current commercial processes, which tolerates the presence of C₁₀₊ aromatics better than those commercial processes. The process may be carried out in the vapor phase or in a preferred embodiment, at least partially in the liquid phase. Alkyl benzenes, such as methylbenzenes and/or ethylbenzenes, and C₁₀₊ aromatics, particularly naphthalene, may be transalkylated to form benzene, toluene, and/or xylenes. The benzene produced may be subjected to further processes, such as transalkylation or methylation, to produce xylenes.

In an aspect, a method for liquid phase transalkylation of aromatic compounds is provided. A feedstock comprising naphthalene and alkyl-substituted benzene can be exposed to a transalkylation catalyst under effective transalkylation conditions to form a transalkylation effluent comprising an alkyl-substituted naphthalene and benzene. Optionally, the feedstock can include at least about 1.0 wt % naphthalene. The mole fraction of aromatic compounds in the liquid phase in the feedstock, relative to the total amount of aromatic compounds in the feedstock, is at least about 0.01 under the effective transalkylation conditions. The catalyst includes at least one of the following: a molecular sieve having an MWW framework molecular sieve with an n value of about 2 to about 50; a molecular sieve corresponding to a Beta polymorph with an n value of about 10 to about 60; and a molecular sieve having a FAU framework with an n value of about 2 to about 400, where n is a molar ratio YO₂ over X₂O₃ in the molecular sieve framework, X is a trivalent element, and Y is a tetravalent element.

### BRIEF DESCRIPTION OF THE FIGURES

FIG. 1 shows results from transalkylation of naphthalene using various catalysts in the presence of a feed containing a mixture of aromatics.

### DETAILED DESCRIPTION

### Overview

In various aspects, systems and methods are provided for performing transalkylation on feeds including alkylated monocyclic aromatic compounds (e.g., alkyl-substituted benzenes) and polycyclic aromatic compounds (e.g., naphthalene). Optionally, the transalkylation can be performed under fixed bed processing conditions. Optionally but preferably, the transalkylation can be performed under conditions where a substantial portion of the transalkylation feed is in the liquid phase.

In some aspects, the transalkylation systems and methods described herein can provide aromatic fluids having a reduced naphthalene concentration and/or processes for producing aromatic fluids having a reduced concentration of naphthalene by transalkylation. Other aspects can provide a process of producing and recovering benzene from C₈ aromatic fluid by transalkylation. The C₈ aromatic fluid typically comprises xylenes and ethylbenzene.

Still other aspects can provide a process of reducing naphthalene concentration in a mixture of aromatic fluids by conversion of the naphthalene to an alkyl-substituted naphthalene and/or a process of removing ethyl-substituted benzene from mixed aromatic fluids. These processes can permit naphthalene-containing aromatic fluid and ethyl-substituted benzene-containing fluid to be converted into products, such as benzene, toluene, and/or xylenes, with enhanced value and/or recovery of by-products for recycling.

An example of a transalkylation process can be a process for making an ethylated polycyclic aromatic compound, the process comprising contacting a mixed aromatic fluid containing a polycyclic aromatic compound and a monocyclic aromatic compound having an ethyl substituent in the presence of an acid catalyst under conditions sufficient to effect transalkylation to form the ethylated polycyclic aromatic compound and a de-ethylated monocyclic aromatic compound. Optionally, the polycyclic compound comprises naphthalene and the monocyclic compound comprises an ethyl-substituted benzene, such as ethylbenzene or methyl-ethyl-benzene. In some aspects, about 20 wt % to about 90 wt % of the polycyclic compound in the mixed aromatic fluid can be converted to the ethylated polycyclic compound. Examples of suitable catalysts can correspond to catalysts including an MWW framework molecular sieve, such as MCM-22, MCM-49, and/or MCM-56. Other types of suitable catalysts can include zeolite Beta (or more generally *BEA framework molecular sieves and/or other Beta polymorphs) and zeolite Y, such as USY (or more generally FAU framework molecular sieves).

In still another aspect, a process for reduction of naphthalene concentration in a mixed aromatic fluid comprises mixing a C₈ aromatic fluid comprising ethylbenzene with a naphthalene-containing aromatic fluid to form a mixed aromatic fluid; contacting the mixed aromatic fluid with an acid catalyst under conditions sufficient to effect transalkylation to form benzene and a naphthalene-depleted mixed aromatic fluid; and separating the benzene from the naphthalene-depleted aromatic fluid. The naphthalene-containing aromatic fluid is exemplified by Aromatic 150™ and Aromatic 200™ Fluids sold by ExxonMobil Chemical Company, although various other commercially available fluids can also include naphthalene.

In one embodiment, preferably from about 20 wt % to about 90 wt % of the polycyclic compound in the naphthalene-containing aromatic fluid is converted to the ethylated polycyclic compound. In one embodiment, the preferred catalyst comprises at least one MWW framework zeolite. Preferably, the naphthalene-depleted fluid comprises less than about 2.0 wt % naphthalene, or less than about 1.0 wt %, or less than about 0.5 wt %, or less than about 0.1 wt %.

The naphthalene concentration in the naphthalene-depleted mixed aromatic fluid may be further reduced by mixing the naphthalene-depleted mixed aromatic fluid with an aromatic fluid comprising ethyl-substituted benzene and contacting the mixture with an acid catalyst under conditions sufficient to effect further transalkylation to form benzene and a naphthalene-depleted mixed aromatic fluid having less than about 1.0 wt %, or less than about 0.5 wt %, or less than about 0.1 wt %; and separating the benzene from the naphthalene-depleted aromatic fluid having less than about 1.0 wt %, or less than about 0.5 wt %, or less than about 0.1 wt %. An example of an aromatic fluid comprising ethyl-substituted benzene can be a C₈ aromatic fluid comprising ethylbenzene.

In yet another example, a process for reducing naphthalene concentration in a mixed aromatic fluid can include contacting an acid catalyst and the mixed aromatic fluid under conditions sufficient to form alkyl-substituted naphthalene and xylenes and/or benzene, wherein the mixed aromatic fluid comprises 1,2,4-trimethylbenzene; 1,2,3-trimethylbenzene; m-cymene; a mixture of alkyl-substituted benzene compounds having from 1 to 4 alkyl substituents, each alkyl substituent having from 1 to 4 carbon atoms and the total carbon atoms in the alkyl-substituted benzene compounds is 10, 11 or 12; naphthalene; and methylnaphthalene. Optionally, about 20 wt % to about 90 wt % of the naphthalene in the mixed aromatic fluid can be converted to alkyl-substituted naphthalene. Optionally, the preferred catalyst comprises at least one MWW framework zeolite. The naphthalene concentration in the product can be less than about 2.0 wt %, or less than about 1.0 wt %, or less than about 0.5 wt %, or less than about 0.1 wt %. In one embodiment, the mixed aromatic fluid comprises Aromatic 150™ or Aromatic 200™ Fluid sold by ExxonMobil Chemical Company.

In still other aspects, a process is provided for selectively transalkylating naphthalene in a mixture of aromatic compounds, the process comprising contacting the mixture of aromatic compounds comprising naphthalene and mononuclear alkylated aromatic compounds with an acid catalyst under conditions sufficient to effect transalkylation of the naphthalene to form alkyl-substituted naphthalene and benzene, wherein the mononuclear alkylated aromatic compounds comprise ethyl-substituted benzene and methylated benzene compounds and wherein the ratio of ethyl-substituted benzene to methylated benzene compounds decreases during the transalkylation process. The ratio of naphthalene concentration in the mixture of aromatic compounds to the naphthalene concentration after transalkylation of the mixture of compounds can range from about 1.2 to about 15, or about 1.2 to about 10, or about 1.5 to about 10, or about 1.5 to about 5. Optionally, about 20 wt % to about 90 wt % of the naphthalene is converted to the alkyl-substituted naphthalene. Optionally, the catalyst can include at least one MWW framework zeolite. Optionally, the naphthalene concentration in the product after transalkylation is less than about 2.0 wt %, or less than about 1.0 wt %, or less than about 0.5 wt %, or less than about 0.1 wt %. The process may further comprise adding mononuclear alkylated aromatic compounds to the product mixture resulting from the initial transalkylation reaction and effecting further transalkylation to further reduce the naphthalene concentration. An example of a mononuclear alkylated aromatic compound is ethylbenzene, which can result in production of benzene and ethylnaphthalene after transalkylation in the presence of an acidic catalyst.

The separation of benzene, toluene, and/or xylenes from any aspect, embodiment, or example described herein may be accomplished by conventional methods including, but not limited to, distillation and extraction.

As used in this specification, the term “framework type” is used in the sense described in the “Atlas of Zeolite Framework Types,” 2001.

The xylene yield, as used herein, is calculated by dividing the total weight of the xylene isomers (para-, meta-, and ortho-xylene) by the total weight of the product stream. The total weight of the xylene isomers can be calculated by multiplying the weight percentage of the xylene isomers, as determined by gas chromatography, by the total weight of the product stream.

Weight of molecular sieve, weight of binder, weight of catalyst composition, weight ratio of molecular sieve over catalyst composition, and weight ratio of binder over catalyst composition are calculated based on calcined weight (at 510° C. in air for 24 hours), i.e., the weight of the molecular sieve, the binder, and the catalyst composition are calculated based on the weight of the molecular sieve, the binder, and the catalyst composition after being calcined at 510° C. in air for twenty-four hours.

The term “aromatic” as used herein is to be understood in accordance with its art-recognized scope which includes alkyl substituted and unsubstituted mono- and polynuclear compounds.

The term “Cₙ” hydrocarbon wherein n is an positive integer, e.g., 1, 2, 3, 4, 5, 6, 7, 8, 9, 10, 11, 12, as used herein means a hydrocarbon having n number of carbon atom(s) per molecular. For example, Cₙ aromatics means an aromatic hydrocarbon having n number of carbon atom(s) per molecule. The term “Cₙ₊” hydrocarbon wherein n is an positive integer, e.g., 1, 2, 3, 4, 5, 6, 7, 8, 9, 10, 11, 12, as used herein means a hydrocarbon having at least n number of carbon atom(s) per molecule. The term “Cₙ₋” hydrocarbon wherein n is an positive integer, e.g., 1, 2, 3, 4, 5, 6, 7, 8, 9, 10, 11, 12, as used herein means a hydrocarbon having no more than n number of carbon atom(s) per molecule.

The term “Cₙ feedstock”, wherein n is a positive integer, e.g., 1, 2, 3, 4, 5, 6, 7, 8, 9, 10, 11, 12, as used herein, means that the Cₙ feedstock comprises greater than 50 wt % (or greater than 75 wt % or greater than 90 wt %) of hydrocarbons having n number of carbon atom(s) per molecule. The term “Cₙ₊ feedstock”, wherein n is a positive integer, e.g., 1, 2, 3, 4, 5, 6, 7, 8, 9, 10, 11, 12, as used herein, means that the Cₙ₊ feedstock comprises greater than 50 wt % (or greater than 75 wt % or greater than 90 wt %) of hydrocarbons having at least n number of carbon atom(s) per molecule. The term “Cₙ₋ feedstock” wherein n is a positive integer, e.g., 1, 2, 3, 4, 5, 6, 7, 8, 9, 10, 11, 12, as used herein, means that the Cₙ₋ feedstock comprises greater than 50 wt % (or greater than 75 wt % or greater than 90 wt %) of hydrocarbons having no more than n number of carbon atom(s) per molecule. The term “Cₙ aromatic feedstock”, wherein n is a positive integer, e.g., 1, 2, 3, 4, 5, 6, 7, 8, 9, 10, 11, 12, as used herein, means that the Cₙ aromatic feedstock comprises greater than 50 wt % (or greater than 75 wt % or greater than 90 wt %) of aromatic hydrocarbons having n number of carbon atom(s) per molecule. The term “Cₙ₊ aromatic feedstock”, wherein n is a positive integer, e.g., 1, 2, 3, 4, 5, 6, 7, 8, 9, 10, 11, 12, as used herein, means that the Cₙ₊ aromatic feedstock comprises greater than 50 wt % (or greater than 75 wt % or greater than 90 wt %) of aromatic hydrocarbons having at least n number of carbon atom(s) per molecule. The term “Cₙ− aromatic feedstock” wherein n is a positive integer, e.g., 1, 2, 3, 4, 5, 6, 7, 8, 9, 10, 11, 12, as used herein, means that the Cₙ− aromatic feedstock comprises greater than 50 wt % (or greater than 75 wt % or greater than 90 wt %) of aromatic hydrocarbons having no more than n number of carbon atom(s) per molecule.

### Feedstock

The aromatic fluid containing naphthalene useful in this process can be derived from a substantially dealkylated feedstock. In some aspects, an aromatic fluid feedstock can include one or more fused-ring polycyclic aromatic compounds, although assemblies of two or more cyclic systems, either single ring cyclics or aromatics or fused systems may also be present. An example of a suitable mixed aromatic fluid can be a fluid that comprises 1,2,4-trimethylbenzene; 1,2,3-trimethylbenzene; m-cymene; a mixture of alkylbenzene compounds having from 1 to 4 alkyl substituents, each alkyl substituent having from 1 to 4 carbon atoms and the alkylbenzene compounds have a total number of carbon atoms ranging from 9 to 12; naphthalene; and methylnaphthalene.

The polycyclic aromatic compound is typically obtained from catalytic reforming operations but may also be obtained from cracking operations, e.g., fluidized bed catalytic cracking (FCC) or moving bed Thermofor catalytic cracking (TCC). Typically, these feed stocks have a hydrogen content of no greater than about 12.5 wt % and API gravity no greater than 25 and an aromatic content no less than 50 wt %.

A substantially dealkylated feedstock is a product that was formerly an alkyl aromatic compound, or mixture of alkyl aromatic compounds, that contained bulky relatively large alkyl group side chains affixed to the aromatic moiety. The dealkylated product is the aromatic compound having no bulky side chain alkyl group. Representative examples of the aromatic compound include phenanthrene, anthracene, dibenzothiophene, fluoroanthene, fluorene, benzothiophene, acenaphthene, biphenyl or naphthalene.

During acid catalyzed cracking and similar reactions, prior dealkylation generally will remove side chains of greater than 5 carbon atoms while leaving behind primarily methyl or ethyl groups on the aromatic compounds. Thus, for purposes of this disclosure, the polycyclic aromatic compounds can include substantially dealkylated aromatic compounds which contain small alkyl groups, such as methyl and sometimes ethyl and the like, remaining as side chains, but with relatively few large alkyl groups, e.g. the C₃ to C₉ groups remaining.

In one embodiment, the polycyclic aromatic feedstock comprises a mixture of polycyclic compounds, dealkylated or substantially dealkylated, which would be found in a refinery by-product stream. Alternatively, the polyaromatic feedstock comprises a relatively pure feed consisting essentially of one type of polycyclic aromatic compound.

Representative examples of suitable polycyclic aromatic refinery by-product derived feedstocks include reformate, light cycle oils and heavy cycle oils from catalytic cracking or pyrolysis processes. Other examples of suitable feedstocks include the liquid product from a delayed or fluid bed coking process, such as a coker gas oil, an aromatics-rich fraction produced by lubricant refining, e.g., furfural extraction. Other sources of suitable feedstocks include a heavy crude fraction obtained by crude fractional distillation.

Specifically, the polycyclic aromatic compound contemplated contains at least 2 cyclic groups and up to 5 cyclic groups. It can be a hydrocarbon containing up to 5 or more benzene rings in any arrangement including fixed benzene rings in linear arrangement. It can be almost entirely or predominantly carbocyclic and can include or be part of a heterocyclic system in which at least one of the cyclic elements of the molecule contains at least one heteroatom such as sulfur, nitrogen and/or oxygen.

In some aspects, the mixture of aromatic compounds may be Aromatic 150™ or Aromatic 200™ fluids sold by ExxonMobil Chemical Company. Aromatic 150™ fluid comprises approximately fifty components with some of the principle components comprising about 1.7 wt % of 1,2,4-trimethylbenzene; about 3.0 wt % of 1,2,3-trimethylbenzene and meta-cumene; a mixture of about 81.6 wt % C-10 to C-12 benzene compounds, having one or more substituents selected from methyl, ethyl, propyl, and butyl; about 8.6 wt % naphthalene; and about 0.3 wt % methylnaphthalene.

Alternatively, the Aromatic 150™ fluid may be distilled at atmospheric pressure to remove about 60 wt % of the lighter components to leave an Aromatic 150™ fluid concentrate that is about 40 wt % of the total material prior to distillation. The Aromatic 150™ fluid concentrate comprises about 20.4 wt % naphthalene.

Aromatic 200™ fluid comprises approximately 25 to 30 components with some of the principle components comprising naphthalene (10 wt %); various alkylnaphthalenes (75 wt %), including 2-methylnaphthalene (26 wt %), 1-methylnaphthalene (13 wt %), 2-ethylnaphthalene (2%), dimethyl naphthalenes (18 wt %), and trimethyl naphthalenes (7 wt %); and the remaining 15 wt % comprises primarily alkylbenzenes, as determined by gas chromatographic analysis.

Aromatic 100™ fluid may also be used. Aromatic 100™ fluid comprises a mixture of components with some of the principle components comprising alkylbenzenes having 9 to 10 carbon atoms, the alkyl groups primarily being methyl and ethyl groups, and some of the principle components comprising propylbenzene (5%), ethylmethylbenzenes (28%), 1,3,5-trimethylbenzene (10%), and 1,2,4-trimethylbenzene (32%).

More generally, a suitable feedstock can include at least about 1.0 wt % polynuclear aromatics, or at least about 2.0 wt %, or at least about 5.0 wt %, or at least about 10.0 wt %, or at least about 20.0 wt %. In some aspects, the feedstock can include at least about 1.0 wt % naphthalene, or at least about 2.0 wt %, or at least about 5.0 wt %, or at least about 10.0 wt %, or at least about 20.0 wt %. Additionally or alternately, the feedstock can include at least about 1.0 wt % alkylated mononuclear aromatics, or at least about 2.0 wt %, or at least about 5.0 wt %, or at least about about 10 wt %. In some aspects, the feedstock can include at least about 1.0 wt % ethylbenzene, or at least about 2.0 wt %, or at least about 5.0 wt %, or at least about 10.0 wt %. Additionally or alternately, the feedstock can include at least about 1.0 wt % of toluene, xylene, or a combination thereof, or at least about 2.0 wt %, or at least about 5.0 wt %, or at least about 10.0 wt %.

### Alkylating Agent

The polycyclic aromatic compound is contacted with an aromatic transalkylating agent, typically, an alkyl-substituted monocyclic aromatic compound. The alkyl-substituted monocyclic aromatic compound can have from one to four short chain alkyl substituents. Preferably, a short chain alkyl substituent contains from 1 to 2 carbon atoms, i.e., methyl and ethyl substituents. Most preferably, the short chain hydrocarbon is ethyl, in which instance the monocyclic aromatic compound is a transethylating agent. Representative examples of transalkylating agents include ethylbenzene, toluene and, ortho-, meta- or para-methylethylbenzene (e.g., o-, m- or p-xylene).

Examples of a source of monocyclic aromatic compound can be a reformate fraction or any other ethyl substituted monocyclic aromatic-rich feed. Specific examples include a reformate from a swing bed or moving bed reformer. Although a most useful source of these monocyclic aromatic compounds is a reformate fraction, other useful sources include pyrolysis gasoline, coker naphtha, methanol-to-gasoline, or other zeolite catalyst olefin or oxygenate conversion process wherein significant aromatics product is obtained.

Another advantage to using the monocyclic aromatic compound as a transalkylating agent, instead of alkylating with an alcohol or alkylhalide, is the resulting conversion of the monocyclic aromatic compound to a gasoline boiling range product when the monocyclic aromatic compound is an ethyl-alkyl-benzene or to benzene when the monocyclic aromatic compound is ethylbenzene. The polyalkylated alkylating agents, having both an ethyl substituent and at least one methyl substituent, are not entirely dealkylated by the reaction. The ethyl substituent is selectively transferred preferentially over a methyl substituent when the ethyl substituent and the methyl substituent are on the same mononuclear aromatic compound. The ethyl substituent is also selectively transferred from ethylbenzene in a mixture also containing methyl- or polymethylbenzene compounds.

An advantage of using the monocyclic aromatic compound as a transalkylating agent, instead of alkylating with an alcohol or alkylhalide, is the resulting conversion of the polyalkylated monocyclic aromatic compound to a gasoline boiling range product or the conversion of ethylbenzene to benzene, which may be separated and recycled.

In one embodiment, the alkylating agent is a C₈ aromatic fluid comprising xylenes and ethylbenzene. In another embodiment, the alkylating agent is a C₈ aromatic fluid consisting primarily of xylenes and ethylbenzene.

The transalkylation of a polycyclic aromatic compound, such as naphthalene, with an alkyl-substituted monocyclic aromatic compound, such as ethylbenzene, is an equilibrium reaction. The equilibrium of the transalkylation is affected by the ratio of the alkyl-substituted monocyclic aromatic compound to naphthalene-containing aromatic compound containing fluid. A higher alkyl-substituted benzene to naphthalene ratio increases the equilibrium concentration of the substituted naphthalene and benzene (or alternatively benzene plus methylated benzene) in the transalkylation process. By controlling the ratio of alkyl-substituted benzene to naphthalene-containing aromatic compound containing fluid, while holding other parameters constant, the concentration of naphthalene in the transalkylation product mixture may be minimized and the concentration of benzene (and/or benzene plus methylated benzene) maximized In one aspect, the naphthalene-containing aromatic fluid is contacted with an acidic catalyst in the presence of an alkyl-substituted benzene containing fluid, wherein the ratio of the alkyl-substituted benzene to naphthalene ranges from about 20 to about 1, preferably about 10 to about 1, and more preferably from about 5 to about 1.

Alternatively, a larger naphthalene to alkyl-substituted benzene ratio increases the equilibrium concentration of benzene (or alternatively benzene plus methylated benzene) formed during the transalkylation. In one aspect, the naphthalene-containing aromatic fluid is contacted with an acidic catalyst in the presence of an alkyl-substituted benzene containing fluid, wherein the ratio of the naphthalene to alkyl-substituted benzene ranges from about 1:1 to preferably about 1:10. One embodiment is a process of recovering benzene from a C₈ aromatic fluid comprising mixing a C₈ aromatic compound fluid with a naphthalene-containing aromatic compound containing fluid in the presence of an acidic catalyst under conditions sufficient to effect transalkylation to form a benzene containing, naphthalene depleted aromatic fluid and separating the benzene from the benzene containing, naphthalene depleted aromatic fluid. A naphthalene depleted aromatic fluid refers to a reduction of more than about 10% of the naphthalene present in the starting naphthalene-containing aromatic fluid. In one embodiment of the process of recovering benzene from a C₈ aromatic compound containing fluid, the C₈ aromatic fluid comprises primarily xylene and ethylbenzene. In another embodiment of the process of recovering benzene from a C₈ aromatic compound containing fluid, the ratio of the naphthalene to alkyl-substituted benzene ranges from about 1 to about 10, preferably from about 1 to about 5.

### Liquid Phase Transalkylation

In this discussion, performing a liquid phase transalkylation reaction corresponds to performing transalkylation under reaction conditions where at least a portion of the aromatic compounds in the reaction environment are in the liquid phase. The mole fraction of aromatic compounds in the liquid phase, relative to the total aromatics, can be at least 0.01, or at least 0.05, or at least 0.08, or at least 0.1, or at least 0.15, or at least 0.2, or at least 0.3, or at least 0.4, or at least 0.5, and optionally up to having substantially all aromatic compounds in the liquid phase.

Due to the difference in volume between gases and liquids, the volume fraction of liquid phase in a reactor can be smaller than the mole fraction. As a rough approximation, the volume of a typical gas phase can be estimated using the ideal gas law. For transalkylation, the volume of a typical aromatic liquid can be estimated by assuming an average molecular weight of about 100-120 g/mol and a liquid phase density of about 0.8 g/ml-0.9 g/ml. Under these assumptions, at a temperature of about 300° C. and a partial pressure of aromatic compounds of about 300 psig, having a liquid mole fraction of about 0.5 would be expected to correspond to having a liquid volume fraction of about 5-10% of the volume of the reaction environment. For a liquid mole fraction of about 0.1, the corresponding liquid volume would be expected to correspond to 0.5-1.0% of the reaction environment. Without being bound by any particular theory, it is believed that formation of even small amounts of a condensed (liquid) phase can substantially alter the nature of a transalkylation reaction environment. Such a liquid phase can potentially form preferentially at surfaces within a reaction environment, such as at the surfaces of catalyst particles. Thus, small amounts of liquid formation can potentially be sufficient to effectively provide liquid phase reaction conditions.

In aspects where the mole fraction of aromatics in the liquid phase is at least 0.4, performing a liquid phase transalkylation can correspond to performing transalkylation under conditions where the liquid phase corresponds to at least about 5% of the total volume of the reaction environment. In such aspects, a continuous liquid phase may optionally be formed in the reaction environment, so that at least 30 vol % of the liquid in the reaction environment forms a single, continuous phase, or at least 50 vol %, or at least 70 vol %. This can be in contrast, for example, to performing transalkylation under trickle-bed conditions, where a plurality of separate liquid phases can form within a fixed catalyst bed. In other aspects, the transalkylation reaction can be performed under trickle-bed conditions.

Generally, for fixed bed and/or trickle bed transalkylation processes, the conditions employed in a liquid phase transalkylation process can include a temperature between 200 to 500° C., or 200 to 340° C., or 230 to 300° C.; a pressure of 1.5 to 10.0 MPa-a, or 1.5 to 8.0 MPa-a, or 1.5 to 7.0 MPa-a, or 3.0 to 8.0 MPa-a, or 3.0 to 7.0 MPa-a, or 3.5 to 6.0 MPa-a; an H₂:hydrocarbon molar ratio of 0 to 20, or 0.01 to 20, or 0.1 to 10; and a weight hourly space velocity (WHSV) for total hydrocarbon feed to the reactor(s) of 0.1 to 100 hr⁻¹, or 1 to 20 hr⁻¹. It is noted that H₂ is not necessarily required during the reaction, so optionally the transalkylation can be performed without introduction of H₂. The feed can be exposed to the transalkylation catalyst under fixed bed conditions, fluidized bed conditions, or other conditions that are suitable when a substantial liquid phase is present in the reaction environment. For other types of reactor configurations such as fluidized bed configurations, a temperature of 270 to 600° C. can be suitable in combination with the pressures, H₂:hydrocarbon molar ratios, and space velocities noted above.

In addition to staying within the general conditions above, the transalkylation conditions can be selected so that a desired amount of the hydrocarbons (reactants and products) in the reactor are in the liquid phase. In various aspects, the transalkylation conditions can be selected so that the mole fraction of liquid phase feed relative to total feed is at least 0.01, or at least 0.1, or any of the other mole fractions noted above.

The lower limits on catalyst activity and on reactive conditions are sufficient to convert at least about 20 wt % and preferably at least about 50 wt % of the polycyclic aromatic compounds in the feed, such as from 20 to 90 wt %. Conversion of the polycyclic aromatic compounds refers to the addition of molecular weight (e.g. ethyl) side chains. The total number of moles of polycyclic aromatic compounds in the product will normally be about the same as the total moles of polycyclic aromatic compounds in the feed to the reactor. The degree of ethylation of the naphthalene, i.e., the degree of naphthalene reduction, ranges from about 20 to 50 wt %, preferably from at least about 40%. The degree of ethylation of the polycyclic aromatic compound, preferably naphthalene, ranges from at least about 10 wt %, more preferably from at least about 20 wt %, even more preferably from at least about 30 wt %, and most preferably from at least about 50 wt %. The degree of ethylation of naphthalene is most preferred when the concentration of naphthalene in the total mixture of aromatic compounds is less than about 1.0 wt %.

### Transalkylation Catalyst

In some aspects, a suitable catalyst for performing (liquid phase) transalkylation can correspond to a molecular sieve having an MWW framework. Examples of MWW framework structure include MCM-22, MCM-49, MCM-56, MCM-36, EMM-10, EMM-13, ITQ-1, ITQ-2, UZM-8, MIT-1, and interlayer expanded zeolites.

The molecular sieve can optionally be characterized based on having a composition with a molar ratio YO₂ over X₂O₃=n, wherein X is a trivalent element, such as aluminum, boron, iron, indium and/or gallium, preferably aluminum and/or gallium, and Y is a tetravalent element, such as silicon, tin and/or germanium, preferably silicon. For an MWW framework molecular sieve, n can be less than about 50, e.g., from about 2 to less than about 50, usually from about 10 to less than about 50, more usually from about 15 to about 40. Optionally, the above n values can correspond to n values for a ratio of silica to alumina in the MWW framework molecular sieve. In such optional aspects, the molecular sieve can optionally correspond to an aluminosilicate and/or a zeolite.

Optionally, the catalyst comprises 0.01 to 5.0 wt %, or 0.01 to 2.0 wt %, or 0.01 to 1.0 wt %, or 0.05 to 5.0 wt %, or 0.05 to 2.0 wt %, or 0.05 to 1.0 wt %, or 0.1 to 5.0 wt %, or 0.1 to 2.0 wt %, or 0.1 to 1.0 wt %, of a metal element of Groups 6-11 (according to the IUPAC Periodic Table). The metal element may be at least one hydrogenation component, such as tungsten, vanadium, molybdenum, rhenium, chromium, manganese, one or more metals selected from Groups 5-11 and 14 of the Periodic Table of the Elements, or mixtures thereof. Optionally, the metal can be selected from Groups 8-10, such as a Group 8-10 noble metal. Specific examples of useful metals are iron, ruthenium, osmium, nickel, cobalt, rhodium, iridium, copper, tin and noble metals such as platinum or palladium. Specific examples of useful bimetallic combinations are bimetallics where Pt is one of the metals, such as Pt/Sn, Pt/Pd, Pt/Cu, and Pt/Rh. In some aspects, the hydrogenation component is palladium, platinum, rhodium, copper, tin, or a combination thereof. The amount of the hydrogenation component can be selected according to a balance between hydrogenation activity and catalytic functionality. For a hydrogenation component including two or more metals (such as a bimetallic hydrogenation component), the ratio of a first metal to a second metal can range from 1:1 to 1:10.

Optionally, a suitable transalkylation catalyst can be a molecular sieve that has a constraint index of 1-12, optionally but preferably less than 3. The constraint index can be determined by the method described in U.S. Pat. No. 4,016,218, which is incorporated herein by reference with regard to the details of determining a constraint index.

Additionally or alternately, a transalkylation catalyst (such as a transalkylation catalyst system) can be used that has a reduced or minimized activity for dealkylation. The Alpha value of a catalyst can provide an indication of the activity of a catalyst for dealkylation. In various aspects, the transalkylation catalyst can have an Alpha value of about 100 or less, or about 50 or less, or about 20 or less, or about 10 or less, or about 1 or less. The alpha value test is a measure of the cracking activity of a catalyst and is described in U.S. Pat. No. 3,354,078 and in the Journal of Catalysis, Vol. 4, p. 527 (1965); Vol. 6, p. 278 (1966); and Vol. 61, p. 395 (1980), each incorporated herein by reference as to that description. The experimental conditions of the test used herein include a constant temperature of 538° C. and a variable flow rate as described in detail in the Journal of Catalysis, Vol. 61, p. 395.

Optionally, a transalkylation catalyst including an MWW framework molecular sieve can further include one or more additional molecular sieves. Examples of additional suitable molecular sieves can include, but are not limited to, molecular sieves with a framework structure having a 3-dimensional network of 12-member ring pore channels. Examples of framework structures having a 3-dimensional 12-member ring are the framework structures corresponding to faujasite (such as zeolite X or Y, including USY), *BEA (such as zeolite Beta), BEC (polymorph C of Beta) CIT-1 (CON), MCM-68 (MSE), hexagonal faujasite (EMT), ITQ-7 (ISV), ITQ-24 (IWR), and ITQ-27 (IWV), preferably faujasite, hexagonal faujasite, and Beta (including all polymorphs of Beta).

For a molecular sieve having the framework structure of Beta and/or its polymorphs, n can be about 10 to about 60, or about 10 to about 50, or about 10 to about 40, or about 20 to about 60, or about 20 to about 50, or about 20 to about 40, or about 60 to about 250, or about 80 to about 250, or about 80 to about 220, or about 10 to about 400, or about 10 to about 250, or about 60 to about 400, or about 80 to about 400.

For a molecular sieve having the framework structure FAU, n can be about 2 to about 400, or about 2 to about 100, or about 2 to about 80, or about 5 to about 400, or about 5 to about 100, or about 5 to about 80, or about 10 to about 400, or about 10 to about 100, or about 10 to about 80.

It may be desirable to incorporate a molecular sieve in the catalyst composition with another material that is resistant to the temperatures and other conditions employed in the transalkylation process of the disclosure. Such materials include active and inactive materials and synthetic or naturally occurring zeolites, as well as inorganic materials such as clays, silica, hydrotalcites, perovskites, spinels, inverse spinels, mixed metal oxides, and/or metal oxides such as alumina, lanthanum oxide, cerium oxide, zirconium oxide, and titania. The inorganic material may be either naturally occurring, or in the form of gelatinous precipitates or gels including mixtures of silica and metal oxides.

Use of a material in conjunction with each molecular sieve, i.e., combined therewith or present during its synthesis, which itself is catalytically active, may change the conversion and/or selectivity of the catalyst composition. Inactive materials suitably serve as diluents to control the amount of conversion so that transalkylated products can be obtained in an economical and orderly manner without employing other means for controlling the rate of reaction. These catalytically active or inactive materials may be incorporated into, for example, alumina, to improve the crush strength of the catalyst composition under commercial operating conditions. It is desirable to provide a catalyst composition having good crush strength because in commercial use, it is desirable to prevent the catalyst composition from breaking down into powder-like materials.

Naturally occurring clays that can be composited with each molecular sieve as a binder for the catalyst composition include the montmorillonite and kaolin family, which families include the subbentonites, and the kaolins commonly known as Dixie, McNamee, Georgia and Florida clays or others in which the main mineral constituent is halloysite, kaolinite, dickite, nacrite or anauxite. Such clays can be used in the raw state as originally mined or initially subjected to calcination, acid treatment or chemical modification.

In addition to the foregoing materials, each molecular sieve can be composited with a binder or matrix material, such as an inorganic oxide selected from the group consisting of silica, alumina, zirconia, titania, thoria, beryllia, magnesia, lanthanum oxide, cerium oxide, manganese oxide, yttrium oxide, calcium oxide, hydrotalcites, perovskites, spinels, inverse spinels, and combinations thereof, such as silica-alumina, silica-magnesia, silica-zirconia, silica-thoria, silica-beryllia, silica-titania, as well as ternary compositions such as silica-alumina-thoria, silica-alumina-zirconia, silica-alumina-magnesia and silica-magnesia-zirconia. It may also be advantageous to provide at least a part of the foregoing porous matrix binder material in colloidal form so as to facilitate extrusion of the catalyst composition.

Each molecular sieve is usually admixed with the binder or matrix material so that the final catalyst composition contains the binder or matrix material in an amount ranging from 5 to 95 wt %, and typically from 10 to 60 wt %.

The particle size and the nature of the conversion catalyst will usually be determined by the type of conversion process which is being carried out, such as: a down-flow, liquid phase, fixed bed process; an up-flow, fixed bed, liquid phase process; an ebullating, fixed fluidized bed liquid or gas phase process; or a liquid or gas phase, transport, fluidized bed process, as noted above, with the fixed-bed type of operation preferred.

Prior to use, steam treatment of the catalyst composition may be employed to minimize the aromatic hydrogenation activity of the catalyst composition. In the steaming process, the catalyst composition is usually contacted with from 5 to 100% steam, at a temperature of at least 260 to 650° C. for at least one hour, specifically 1 to 20 hours, at a pressure of 100 to 2590 kPa-a.

The hydrogenation component can be incorporated into the catalyst composition by any convenient method. Such incorporation methods can include co-crystallization, exchange into the composition (to the extent a Group 13 element, e.g., aluminum, is in the molecular sieve structure), liquid phase and/or vapor phase impregnation, or mixing with the molecular sieve and binder, and combinations thereof. For example, in the case of platinum, a platinum hydrogenation component can be incorporated into the catalyst by treating the molecular sieve with a solution containing a platinum metal-containing ion. Suitable platinum compounds for impregnating the catalyst with platinum include chloroplatinic acid, platinous chloride and various compounds containing the platinum ammine complex, such as Pt(NH₃)₄Cl₂.H₂O. Palladium can be impregnated on a catalyst in a similar manner

Alternatively, a compound of the hydrogenation component may be added to the molecular sieve when it is being composited with a binder, or after the molecular sieve and binder have been formed into particles by extrusion or pelletizing. Still another option can be to use a binder that is a hydrogenation component and/or that includes a hydrogenation component.

After treatment with the hydrogenation component, the molecular sieve is usually dried by heating at a temperature of 65 to 160° C., typically 110 to 143° C., for at least 1 minute and generally not longer than 24 hours, at pressures ranging from 100 to 200 kPa-a. Thereafter, the molecular sieve may be calcined in a stream of dry gas, such as air or nitrogen, at temperatures of from 260 to 650° C. for 1 to 20 hours. Calcination is typically conducted at pressures ranging from 100 to 300 kPa-a.

In addition, prior to contacting the catalyst composition with the hydrocarbon feed, the hydrogenation component can optionally be sulfided. This is conveniently accomplished by contacting the catalyst with a source of sulfur, such as hydrogen sulfide, at a temperature ranging from about 320 to 480° C. The source of sulfur can be contacted with the catalyst via a carrier gas, such as hydrogen or nitrogen. Sulfiding per se is known and sulfiding of the hydrogenation component can be accomplished without more than routine experimentation by one of ordinary skill in the art in possession of the present disclosure.

### EXAMPLE

Conversion of C₉₊ aromatics under fixed bed transalkylation conditions was performed on a mixture of aromatics. The feed used for the transalkylation processes is shown in Table 1.

TABLE 1
Feed for Naphthalene Transalkylation

| Component | Concentration, wt % |
|---|---|
| Trimethylbenzenes | 2.6 |
| Methylethylbenzenes | 0.0 |
| Indane | 1.3 |
| Diethylbenzenes | 1.9 |
| Methylpropylbenzenes | 7.8 |
| Dimethylethylbenzenes | 34.1 |
| Tetramethylbenzenes | 13.8 |
| Methylindanes | 9.6 |
| Naphthalene | 8.3 |
| Methylnaphthalene | 0.0 |
| Bottoms | 20.6 |

As can be seen from Table 1, the feed is a complex mixture of C₉-C₁₁ aromatics with the majority being C₁₀ aromatics. Dimethylethylbenzene is one of the key components in the feed. The feed also contains about 8% naphthalene.

The feed was exposed to various catalysts at 275° C., a weight hourly space velocity of 1.5 hr⁻¹, and a pressure of about 500 psig (˜3.4 MPa-g) over a period of days. These conditions are believed to provide a suitable mole fraction of liquid in the reaction environment for liquid phase transalkylation. The liquid mole fraction of feedstock in the reaction environment is believed to be at least 0.1. Each catalyst corresponded to a bound zeolite. A first catalyst corresponded to MCM-49 bound with alumina (80 wt % zeolite, 20 wt % alumina). A second catalyst corresponded to MCM-22 bound with alumina (65 wt % zeolite, 35 wt % alumina). A third catalyst corresponded to mordenite bound with alumina (65 wt % mordenite, 35 wt % alumina). A fourth catalyst was also a bound mordenite catalyst, but with silica instead of alumina as the binder.

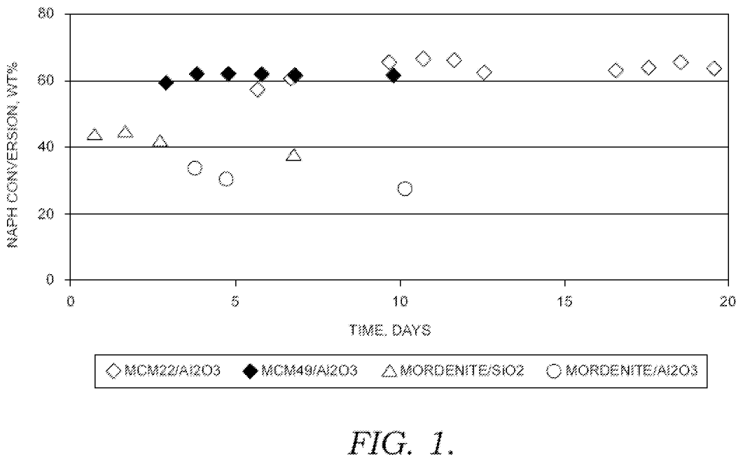

FIG. 1 shows the amount of naphthalene conversion as measured over time during the transalkylation runs. As shown in FIG. 1, the naphthalene conversion for the MWW framework catalysts (MCM-22, MCM-49) was stable during a 2-3 week exposure of the feedstock to both types of catalyst. The conversion of naphthalene in the feed was about 60 wt %, which is believed to be close to the equilibrium conversion amount based on the nature of the feed. By contrast, the mordenite catalysts showed an initially lower activity of about 40 wt % or less conversion, and that activity appeared to drop further during the course of the transalkylation run. The data in FIG. 1 suggest that MWW framework catalysts can provide an unexpectedly improved activity as well as stable activity during liquid phase transalkylation of naphthalene.

While various embodiments have been described and illustrated, it is to be understood that this disclosure is not limited to the particulars disclosed and extends to all equivalents within the scope of the claims. Unless otherwise stated, all percentages, parts, ratios, etc. are by weight. Unless otherwise stated, a reference to a compound or component includes the compound or component by itself as well as in combination with other elements, compounds, or components, such as mixtures of compounds. Further, when an amount, concentration, or other value or parameter is given as a list of upper preferable values and lower preferable values, this is to be understood as specifically disclosing all ranges formed form any pair of an upper preferred value and a lower preferred value, regardless of whether ranges are separately disclosed. All patents, test procedures, and other documents cited herein, including priority documents, are fully incorporated by reference to the extent such disclosure is not inconsistent and for all jurisdictions in which such incorporation is permitted.

What is claimed is:

1. A method for liquid phase transalkylation of aromatic compounds, comprising:
exposing an aromatic feedstock comprising at least about 1.0 wt % naphthalene and alkyl-substituted benzene to a transalkylation catalyst under effective transalkylation conditions to form a transalkylation effluent comprising an alkyl-substituted naphthalene and benzene;
wherein a mole fraction of aromatic compounds in the liquid phase, relative to the total amount of aromatic compounds in the feedstock, is at least about 0.01 under the effective transalkylation conditions; and
wherein the transalkylation catalyst comprises at least one of the following:
a first molecular sieve having an MWW framework with an n value of about 2 to about 50;
a second molecular sieve corresponding to a Beta polymorph with an n value of about 10 to about 60; and
a third molecular sieve having a FAU framework with an n value of about 2 to about 400;
where n is a molar ratio YO₂ over X₂O₃ in the framework of the first, second, and third molecular sieves, X is a trivalent element, and Y is a tetravalent element.

2. The method of claim 1, wherein the transalkylation catalyst further comprises 0.01 wt % to 5 wt % of a metal from Groups 5-11 and 14 supported on the transalkylation catalyst.

3. The method of claim 2, wherein the metal from Groups 5-11 and 14 is selected from the group consisting of Pd, Pt, Ni, Rh, Sn, or a combination thereof.

4. The method of claim 1, wherein the MWW framework of the first molecular sieve is selected from the group consisting of MCM-22, MCM-49, MCM-56, or a combination thereof.

5. The method of claim 1, wherein the transalkylation catalyst further comprises a binder.

6. The method of claim 1, wherein the mole fraction of aromatic compounds in the liquid phase in the feedstock, relative to the total amount of aromatic compounds in the feedstock, is at least about 0.1 under the effective transalkylation conditions.

7. The method of claim 1, wherein the effective transalkylation conditions comprise a temperature of about 200 to about 500° C.; a total pressure of about 10 MPa-g or less; or a combination thereof.

8. The method of claim 1, wherein the effective transalkylation conditions comprise a molar ratio of H₂ to hydrocarbons in the feedstock of about 0.01 to about 10.

9. The method of claim 1, wherein the aromatic feedstock comprises at least 5.0 wt % naphthalene.

10. The method of claim 1, wherein the aromatic feedstock comprises at least 1.0 wt % alkyl-substituted benzene.

11. The method of claim 1, wherein the alkyl-substituted benzene comprises ethyl-substituted benzene, the aromatic feedstock comprising at least 1.0 wt % ethyl-substituted benzene.

12. A method for liquid phase transalkylation of aromatic compounds, comprising:
exposing a feedstock comprising at least about 1.0 wt % naphthalene and ethylbenzene to a transalkylation catalyst under effective transalkylation conditions to form a transalkylation effluent comprising ethylnaphthalene and benzene;
wherein a mole fraction of aromatic compounds in the liquid phase in the feedstock, relative to the total amount of aromatic compounds in the feedstock, is at least about 0.01 under the effective transalkylation conditions; and
wherein the transalkylation catalyst comprises at least one of the following:
a first molecular sieve having an MWW framework with an n value of about 2 to about 50;
a second molecular sieve corresponding to a Beta polymorph with an n value of about 10 to about 60; and
a third molecular sieve having a FAU framework with an n value of about 2 to about 400;
where n is a molar ratio YO₂ over X₂O₃ in the framework of the first, second, and third molecular sieves, X is a trivalent element, and Y is a tetravalent element.

13. The method of claim 12, wherein the transalkylation catalyst further comprises 0.01 wt % to 5 wt % of a metal from Groups 5-11 and 14 supported on the catalyst.

14. The method of claim 13, wherein the metal from Groups 5-11 and 14 is selected from the group consisting of Pd, Pt, Ni, Rh, Sn, or a combination thereof.

15. The method of claim 12, wherein the MWW framework of the first molecular sieve is selected from the group consisting of MCM-22, MCM-49, MCM-56, or a combination thereof.

16. The method of claim 12, wherein from about 20 wt % to about 90 wt % of the naphthalene in the feedstock is converted to ethylnaphthalene.

17. The method of claim 12, wherein a naphthalene concentration in the transalkylation effluent is less than about 1.0 wt %.

18. The method of claim 12, wherein the feedstock further comprises toluene, xylene, or a combination thereof.

19. The method of claim 12, wherein the feedstock comprises at least 5.0 wt % naphthalene.
